(19) United States
(12) Patent Application Publication
(10) Pub. No.: US 2019/0336707 A1
(43) Pub. Date: Nov. 7, 2019

(54) FLUID INJECTION PRESSURE CONTROL SYRINGE

(71) Applicant: Huaiqing YU, XINXIANG (CN)

(72) Inventor: Huaiqing YU, XINXIANG (CN)

(21) Appl. No.: 16/516,639

(22) Filed: Jul. 19, 2019

Related U.S. Application Data
(63) Continuation of application No. PCT/CN2018/000028, filed on Jan. 24, 2018.

(30) Foreign Application Priority Data
Mar. 16, 2017 (CN) 201710152487.9

(51) Int. Cl. A61M 5/48; A61M 5/315; A61M 5/31; A61M 5/19

(52) U.S. Cl. CPC A61M 5/486; A61M 5/31513; A61M 2205/702; A61M 5/19; A61M 2205/583; A61M 5/3129

(57) ABSTRACT

A fluid injection pressure control syringe is a convenient device to reflect changes of fluid pressure inside the syringe barrel or close communicating vessel by utilizing the change of compression amplitude of a spring with certain elasticity coefficient, including syringe barrel, pushrod with sealing gasket, spring, indication pole with little sealing gasket on the surface of indication pole, there is pressure scale or pressure range information, and hole cap. The main physical principle A, P=(F₁+F₂)/S; Physical principle B, F₁=K(L₀−L). P is the pressure of the fluid in the syringe barrel; F₁ is the spring compression reaction force; F₂ is the sliding friction force between sealing gasket and sub-barrel wall (negligible when micro); S is the cross-sectional area of the sub-barrel inner lumen. K is the elastic coefficient of the spring, L₀ is the original length of the spring, and L is the length after compression.

### CROSS-REFERENCE TO RELATED APPLICATIONS

This application is a continuation of International Patent Application No. PCT/CN2018/000028 with a filing date of Jan. 24, 2018, designating the United States, now pending, and further claims priority to Chinese Patent Application No. 201710152487.9 with a filing date of Mar. 16, 2017. The content of the aforementioned applications, including any intervening amendments thereto, are incorporated herein by reference.

### TECHNICAL FIELD

The invention relates to the field of medical devices, in particular to fluid injection pressure control.

### BACKGROUND OF THE PRESENT INVENTION

With the continuous advancement of medical technology, it is often necessary to use a syringe to inject fluid substances (air, liquid, body filling materials, etc.) in medical clinical practice or medical treatment. In many medical departments, when injecting the corresponding fluid, it is necessary to control the fluid injection pressure, or to evaluate whether the medical procedure is safe or effective by the fluid injection pressure.

In airway anesthesia management, such as commonly used with cuffed endotracheal tube, inflatable laryngeal mask, endobronchial tube, tracheostomy tube, etc., which need to inflate balloon with common syringe and seal the airway. And need ensure that the pressure inside the balloon is within a certain range, cannot be too high or too low (if too low, cannot be completely sealed, too high will damage the airway wall). Currently clinical staff control the balloon pressure mainly by two methods: one is using a common syringe to inject air into the balloon, then use the pressure garge to check, if the pressure out of the standard range adjust again. The second is after the first inflation with common syringe, feeling the pressure through finger touching the pilot which is connected with balloon, and then judge whether the pressure is standard. The shortcoming is that the first method needs to be reconnected to another checking equipment, which is cumbersome and repeatedly adjusted to waste valuable time; Second method is judged by personal finger feeling, strong randomness, further adjustment can be repeating inflation or deflation, cumbersome and waste of time.

In the field of nerve block therapy, the ideal drug injection site is the peripheral area of the nerve bundle. When the needle tip penetrates into the nerve bundle or the superficial injection, it will cause nerve damage, some cases will cause permanent neurological deficit. Clinical studies have confirmed that the pressure requirement is greater than 15 psi when injected into nerve bundle; the needle tip touch nerve bundle superficial injection pressure requirement of is the majority (97%) at 15 psi; the pressure requirement is less than 15 psi when the needle tip is proximal to nerve bundle (around 1 mm). Studies have confirmed that a reasonable nerve peripheral injection site pressure requirement is no more than 4 psi. At present, some clinical practice is to connect a pressure monitoring device to the syringe and under the guide of ultrasound close to the nerve bundle, and judge right injection position by the injection pressure. The shortcoming of this approach is that the operation is cumbersome, time consuming, and costly.

In the field of minimally invasive interventional therapy, the balloon expansion pressure pump is generally used, which looks like a large syringe. The front part has a pointer pressure gauge or a digital pressure gauge. The downside is that production costs are relatively high.

### SUMMARY OF THE INVENTION

The purpose of the present invention is to design a fluid injection pressure control syringe which is more convenient, quick, simple structure, low cost, and is designed to solve the existing problems in the prior tech.

Technical measures taken to implement the invention:

The fluid injection pressure control syringe, comprises syringe barrel (at front end of the syringe there is a sub-barrel as communicating vessel with the main barrel), pushrod with sealing gasket, spring, little sealing gasket with indication pole (with scale information on the indication pole surface), hole-cap (fastened at the end of the sub-barrel).

There is a sub-barrel at the front art of the syringe body; the main barrel and the sub-barrel is communicating vessel structure.

A metal or non-metallic spring with a certain coefficient of elasticity is placed in the sub-barrel of the syringe.

Little sealing gasket with indication pole in the sub-barrel, be used for sealing the fluid in the sub-barrel and used for pressuring the spring and bounding the spring in the sub-barrel.

When the little sealing gasket be pressured by the fluid from the syringe mam barrel, the little sealing gasket with indication pole will slide and compress the spring in the sub-barrel; and at the same time, the indication pole can lean out from the hole on the cap. As the fluid pressure increases, the spring compression amplitude increases, and lean-out amplitude of the indication pole also increases.

The main physical principle A, P=(F1+F2)/S; Physical principle B, F1=K(L0−L). P is the pressure of the fluid in the syringe barrel; F1 is the spring compression reaction force; F2 is the sliding friction force between sealing gasket and the sub-barrel wall (negligible when micro); S is the cross-sectional area of the sub-barrel inner lumen K is the elastic coefficient of the spring, L0 is the original length of the spring, and L is the length after compression.

### BRIEF DESCRIPTION OF THE DRAWINGS

FIG. 1 is a schematic view showing the appearance of a fluid injection pressure control syringe in a natural state;

FIG. 2 is a schematic cross-sectional structural view of a fluid injection pressure control injector in a natural state;

FIG. 3 is an enlarged schematic view showing the cross-sectional structure of the front portion of a fluid injection pressure control injector in a natural state;

FIG. 4 is a schematic view showing the appearance of a fluid injection pressure control injector after the internal fluid pressure is increased;

FIG. 5 is a schematic cross-sectional structural view of a fluid injection pressure control injector after the internal fluid pressure is increased;

FIG. 6 is an enlarged schematic view showing the cross-sectional structure of the front portion of a fluid injection pressure control injector after the internal fluid pressure is increased;

FIG. 7 is schematic and cross-sectional view showing gasket and indication pole, spring, hole-cap assembly structure in a natural state;

FIG. 8 is schematic and cross-sectional view showing the structure and structure of gasket and indication pole, spring, hole-cap after internal fluid pressure is increased;

FIG. 9 is a schematic view showing a scheme for realizing the surface pressure scale of indication pole;

FIG. 10 is schematic and cross-sectional view showing the appearance and structure of the main body of a fluid injection pressure control syringe.

### DETAILED DESCRIPTION OF THE EMBODIMENTS

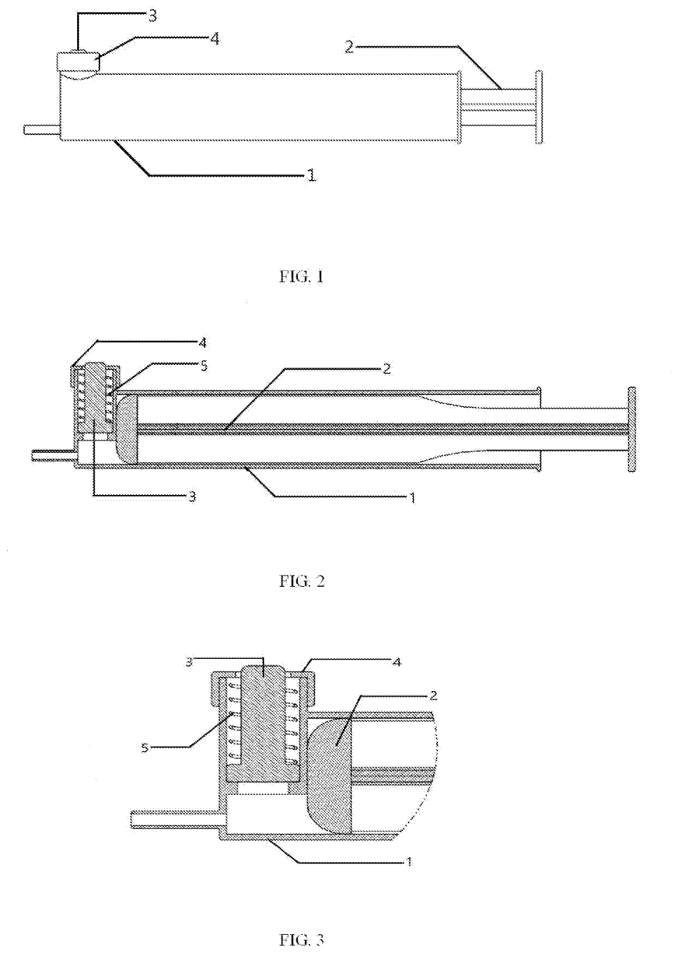

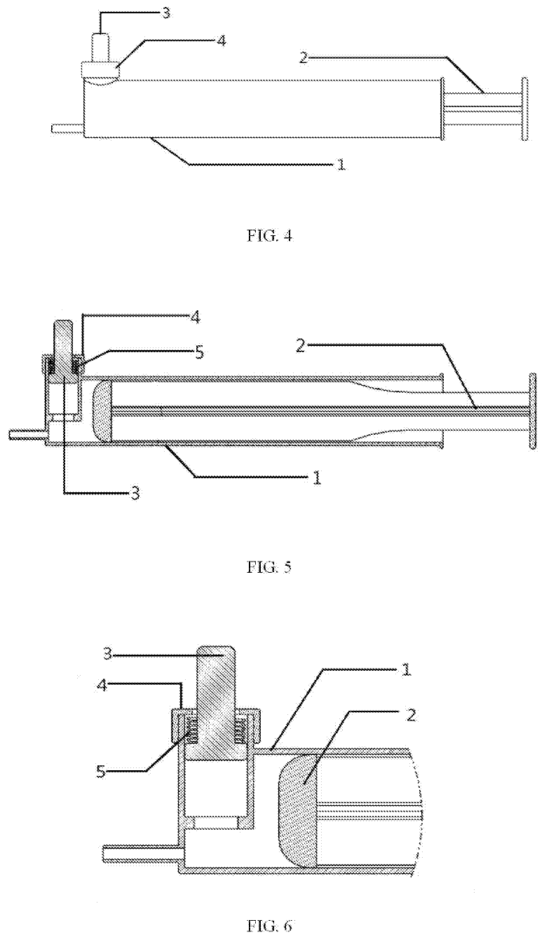

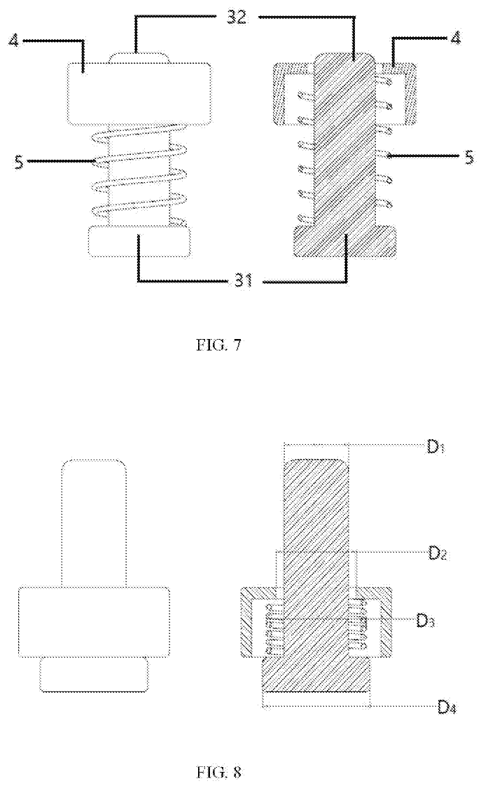

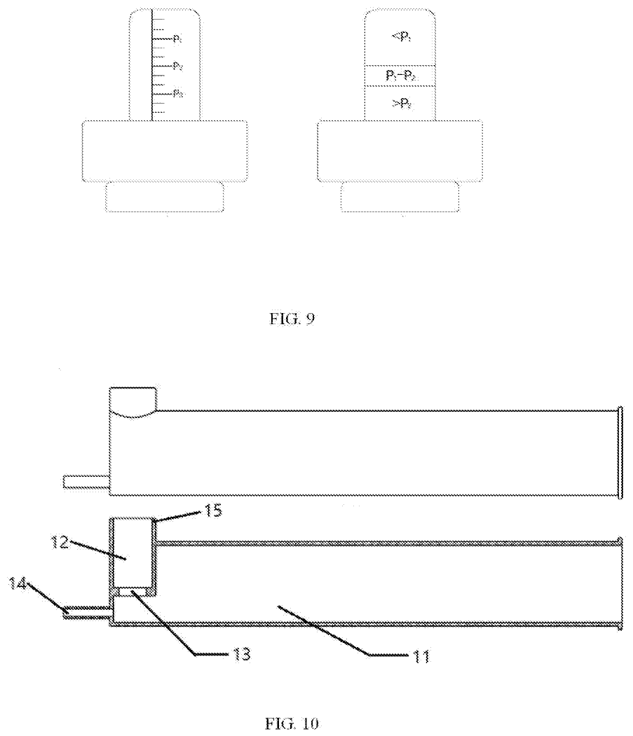

FIG. 1 and FIG. 2 present a schematic view of the appearance and structure of a fluid injection pressure control syringe provided of the invention, including syringe main body 1, pushrod with sealing gasket 2, indication pole with little sealing gasket 3 (the surface of the indication pole containing pressure scale or pressure range information is shown in FIG. 9), hole-cap 4, spring 5; as shown in FIG. 10, the syringe main body 1 includes a main barrel 11 and a sub-barrel 12 at the front end, and the main barrel 11 and the sub-barrel 12 constructing a commutating vessels through the hole 13 at bottom of the sub-barrel 12; as shown in FIG. 3, FIG. 7, FIG. 10, indication pole with little sealing gasket 3 is placed in sub-barrel 12, and the little sealing gasket 31 well sealing the sub-barrel 12, are reasonably fitted to ensure that the fluid from the main barrel 11 does not overflow through the sub-barrel 12 within the design pressure range and the sliding friction force between little sealing gasket 31 and the sub-barrel 12 is within the design range; as shown in FIG. 7, spring 5 sleeve on indication pole 32, and is bounded by hole-cap 4 and little sealing gasket 31; as shown in FIG. 7, indication pole 32 can lean out through the hole of hole-cap 4; as shown in FIG. 7 FIG. 8, the diameter D1 of indication pole 32, the diameter D2 of the hole of hole-cap, the diameter D3 of spring, the diameter D4 of little sealing gasket 31, the relationship D1<D2<D3<D4; as shown in FIG. 1 and FIG. 10, hole-cap 4 is fastened to the top part 15 of the sub-barrel 12; as shown in FIG. 2 FIG. 10, pushrod with sealing gasket 2 is placed in the main barrel 11 of the syringe main body 1, after being subjected to an external force, it can be moving back or forward in the main barrel 11.

The main physical principle A, P=(F1+F2)/S; Physical principle B, F1=K(L0−L). P is the pressure of the fluid in the syringe barrel force to little sealing gasket 31; F1 is spring 5 compression reaction force; F2 is the sliding friction force between little sealing gasket 31 and the sub-barrel 12 wall (negligible when micro); S is the cross-sectional area of the sub-barrel 12 inner lumen. K is the elastic coefficient of spring 5, L0 is the original length of spring 5, and L is the length after compression.

The relation for the specific performance: Push the sealing gasket with pushrod 2 forward, fluid pressure in the syringe main barrel rises, little gasket 31 by action of fluid pressure, little sealing gasket with indication pole 3 slid and compress spring 5, indication pole 32 lean out the hole of hole-cap 4.

In use, pull the pushrod 2, suck the desired fluid through suction port 14 (FIG. 10), connect the suction port 14 with the respective device or instrument firmly; then push pushrod 2, observe the rising of indication pole 32, and checking the pressure scale information on indication pole 32 (FIG. 9), according to the clinical application judge the standard.

We claim:

1. A new fluid injection pressure control syringe, characterized by changing in a compression amplitude of a metal or non-metal spring with certain elastic coefficient, to reflect fluid pressure change in a syringe barrel or communicating vessels closed to the syringe barrel, comprising a syringe barrel, a pushrod with a sealing gasket, a spring, an indication pole with little sealing gasket, and a hole-cap, wherein a surface of the indication pole contains pressure scale or pressure range information;
a main physical principle A, P=(F₁+F₂)/S; a physical principle B, F₁=K(L₀−L), wherein P is a pressure of fluid in the syringe barrel; F₁ is a spring compression reaction force; F₂ is a sliding friction force between the sealing gasket and a wall of a sub-barrel, wherein the sliding friction force is negligible when micro: S is a cross-sectional area of a sub-barrel inner lumen; K is an elastic coefficient of the spring, L₀ is an original length of the spring, and L is a length after compression.

2. The fluid injection pressure control syringe according to claim 1, wherein at a front part of the syringe is provided with the sub-barrel, the sub-barrel of the syringe and a main barrel of the syringe are communicating vessels relationship.

3. The fluid injection pressure control syringe according to claim 1, wherein there is a metallic or non-metallic spring with a coefficient of elasticity placed in the sub-barrel at a front part of the syringe.

4. The fluid injection pressure control syringe according to claim 1, wherein the indication pole with a sealing gasket is placed in the sub-barrel of the syringe, configure to seal fluid from a main barrel without overflowing and compressing and bounding the spring in the sub-barrel.

5. The fluid injection pressure control syringe according to claim 1, wherein the hole-cap is disposed at an end of the sub-barrel of the syringe; the hole-cap is provided with a suitable size hole; a diameter of the hole is larger than a diameter of the indication pole and smaller than the diameter of the spring.

6. The fluid injection pressure control syringe according to claim 1, wherein a cross-sectional diameter of the sealing gasket with indication pole is bigger than a diameter of the spring.

7. The fluid injection pressure control syringe according to claim 1, wherein when the sealing gasket with the indication pole which located in the sub-barrel receives fluid pressure from a syringe main barrel, the sealing gasket with the indication pole is able to slid in the sub-barrel and compress the spring; the indication pole is able to lean out through a hole on the hole-cap; as the fluid pressure in the syringe barrel increases, a compression ratio of the spring increases, and a lean-out part of the indication pole also increased.
